(19) United States
(12) Patent Application Publication
(10) Pub. No.: US 2022/0139567 A1
(43) Pub. Date: May 5, 2022

(54) METHODS FOR MODELING INFECTIOUS DISEASE TEST PERFORMANCE AS A FUNCTION OF SPECIFIC, INDIVIDUAL DISEASE TIMELINES

(71) Applicant: The Boeing Company, Chicago, IL (US)

(72) Inventors: Lindsay Leigh Waite Jones, Madison, AL (US); Robert M. Lawton, Huntsville, AL (US); Stephen Paul Jones, Snoqualmie, WA (US); Thomas Robert Austin, Rancho Palos Verdes, CA (US); Jason Wesley Armstrong, Fig Tree Pocket (AU)

(21) Appl. No.: 17/516,686

(22) Filed: Nov. 1, 2021

Related U.S. Application Data
(60) Provisional application No. 63/108,271, filed on Oct. 30, 2020.

(51) Int. Cl. G16H 50/80; G16H 50/50; G16H 50/20; G06N 7/00

(52) U.S. Cl. CPC G16H 50/80; G06N 7/005; G16H 50/20; G16H 50/50

(57) ABSTRACT

Aspects of the disclosure provide solutions for modeling an efficacy of disease screening and testing strategies for an infectious disease. Examples include: identifying events for a disease timeline for the infectious disease, creating a model of test sensitivity as a function of the events, adaptively mapping the events to characteristics of the infectious disease unique to a simulated infected person, based at least on adaptively mapping, creating a unique disease timeline for the simulated infected person, and creating a numerical function specific to the unique disease timeline to model sensitivity as a function of the unique disease timeline.

### CROSS-REFERENCE TO RELATED APPLICATION

This application claims the benefit of and priority to U.S. Provisional Application No. 63/108,271, entitled “Methods for Modeling Infectious Disease Test Performance as a Function of Specific, Individual Disease Timelines”, filed Oct. 30, 2020, which is incorporated by reference herein in its entirety.

### BACKGROUND

The travel industry, and airlines in particular, have faced uncertainty and risk since the beginning of the Covid-19 pandemic. To safely expand international air travel, data-driven, risk-based approaches have been used when assessing the appropriate passenger screening protocols for cross-border travel to minimize disease translocation risks. These conventional approaches attempt to determine the efficacy of various screening strategies to detect infections of an infectious disease with less than effective results.

In light of the foregoing, there is a need for an improved method of modeling infectious disease test performance.

### SUMMARY

The disclosed examples are described in detail below with reference to the accompanying drawing figures listed below. The following summary is provided to illustrate examples or implementations disclosed herein. It is not meant, however, to limit all examples to any particular configuration or sequence of operations.

The present disclosure relates to methods for modeling an efficacy of disease screening and testing strategies for an infectious disease by adapting a function describing performance of an infectious disease test over time to an individual's specific disease timeline rather than using a generic function that is applied to all individuals. As such, modeling the efficacy of the infectious disease accounts for variability in events along a timeline of the infectious disease enabling a more accurate model of a performance of the test.

Examples provided herein include a method for creating unique disease timelines for modeling an efficacy of a screening and testing strategy for an infectious disease. An example includes identifying events for a disease timeline for the infectious disease, the events comprising disease exposure, a symptom onset, a severe symptom onset, and an end of contagious period; creating a model of test sensitivity as a function of the events; adaptively mapping the events to characteristics of the infectious disease unique to a simulated infected person; based at least on adaptively mapping, creating a unique disease timeline for the simulated infected person; and creating a numerical function specific to the unique disease timeline to model sensitivity as a function of the unique disease timeline.

The features, functions, and advantages can be achieved independently in various embodiments of the present disclosure or may be combined in yet other embodiments.

### BRIEF DESCRIPTION OF THE DRAWINGS

These and other features, aspects, and advantages of the present disclosure will become better understood with regard to the following description, appended claims, and accompanying drawings where:

FIG. 1 is a block diagram of a computing device 100 suitable for implementing various aspects of the disclosure in accordance with an example.

FIG. 2 is a flowchart 200 illustrating a method in accordance with an example.

FIG. 3 is a diagram 300 illustrating a distribution for time to symptom onset for a simulated infected person in accordance with an example;

FIG. 4 is a diagram 400 illustrating time to onset of severe symptoms and time to end of the contagious period are modeled as a function of time from symptom onset in accordance with an example.

FIG. 5 is a diagram 500 illustrating a probability density of time from symptom onset to end of contagious in accordance with an example.

FIG. 6 is a diagram 600 illustrating an end of a contagious period modeled as a function of time from symptom onset in accordance with an example.

FIG. 7 shows screening results comparison for metrics of interest in accordance with an example.

FIG. 8 is a block diagram of a computing device suitable for implementing various aspects of the disclosure in accordance with an example.

Corresponding reference characters indicate corresponding parts throughout the drawings in accordance with an example.

### DETAILED DESCRIPTION

The various examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all implementations.

The foregoing summary, as well as the following detailed description of certain implementations will be better understood when read in conjunction with the appended drawings. As used herein, an element or step recited in the singular and preceded by the word “a” or “an” should be understood as not necessarily excluding the plural of the elements or steps. Further, references to an implementation or an example are not intended to be interpreted as excluding the existence of additional examples that also incorporate the recited features. Moreover, unless explicitly stated to the contrary, examples “comprising” or “having” an element or a plurality of elements having a particular property could include additional elements not having that property.

Aspects and implementations disclosed herein are directed to methods and systems for creating unique disease timelines for modeling an efficacy of a screening and testing strategy for an infectious disease. An example includes identifying events for a disease timeline for the infectious disease, the events comprising disease exposure, a symptom onset, a severe symptom onset, and an end of contagious period; creating a model of test sensitivity as a function of the events; adaptively mapping the events to characteristics of the infectious disease unique to a simulated infected person; based at least on adaptively mapping, creating a unique disease timeline for the simulated infected person; and creating a numerical function specific to the unique disease timeline to model sensitivity as a function of the unique disease timeline.

To safely expand international air travel, a data-driven, risk-based approach is needed when assessing appropriate passenger screening and testing protocols for cross-border travel to minimize disease translocation risks from travel between different locations (e.g., cities and countries). Aspects of the disclosure model an efficacy of disease screening and testing strategies for an infectious disease by adapting a function describing performance of an infectious disease test over time to an individual's specific disease timeline rather than using a generic function that is applied to all individuals in conventional modeling. As such, in the exampled provided herein, modeling the efficacy of the infectious disease accounts for variability in events along a timeline of the infectious disease enabling a more accurate model of a performance of the test. For example, a sensitivity of a COVID-19 test performed is a function of each individual simulated infected person's unique disease timeline. Cubic splines are used to create an individual test performance trajectory tied to specific events in the simulated infected person's unique disease timeline, which represents a progression of a disease as a function of time. The function is then used to determine a unique probability of a positive test result for each time point (e.g., event) in the simulated infected person's disease trajectory. As a result, the present disclosure provides a measure of performance for a diagnostic test that is specific to an infected individual's unique disease timeline.

The modeling of various implementations described herein can be used to estimate screening method performance for a variety of different locations around the globe and for different screening strategies. It should be appreciated that the model can be updated, such as when new information about an infectious disease or test performance becomes available. For example, the model framework and model parameters can be updated or modified based on updated test results.

Further, the modeling of various implementations described herein provide governments, regulatory bodies, and medical experts with validated findings to help inform decisions on safe travel around the world. The efficacy metrics described herein are obtained through a Monte Carlo simulation-based analysis comparing the efficacy of several different passenger screening strategies for travel between countries with various COVID-19 prevalence levels to determine the efficacy of introducing a screening strategy to detect infections at various phases of a timeline for the infectious disease infection timeline. Each screening strategy is based on one or more COVID-19 Reverse Transcription Polymerase Chain Reaction (RT-PCR) tests conducted at varying time points. Further, while various examples are described in connection with COVID-19, aspects of the disclosure can be implemented in connection with other infectious diseases and tests.

Aspects of the disclosure have a technical effect of improved screening and testing strategies for an infectious disease. That is, unlike using a generic function that is applied to all individuals as conventional screening and testing strategies have done, the disclosed systems and methods adapt a function describing test performance over time to each simulated infected person's specific disease timeline providing a more accurate representation of data. Therefore, a model is able to account for variability in events of the disease timeline, which allows for a more accurate model of test performance. In this manner, when a processor is programmed to perform the operations described herein, the processor is used in an unconventional way, and allows for the more efficient and robust generation of models for infectious disease test performance. Furthermore, the systems and methods described herein exhibit a technical effect for minimizing disease translocation risks from travel between different locations (e.g., cities and countries).

Reference is now made to the drawings, wherein like reference numerals are used to refer to like elements throughout. In the following description, for purposes of explanation, numerous specific details are set forth in order to provide a thorough understanding thereof. It may be evident, however, that the novel implementations can be practiced without these specific details. In other instances, well known structures and devices are shown in block diagram form in order to facilitate a description thereof. The intention is to cover all modifications, equivalents, and alternatives falling within the spirit and scope of the claimed subject matter.

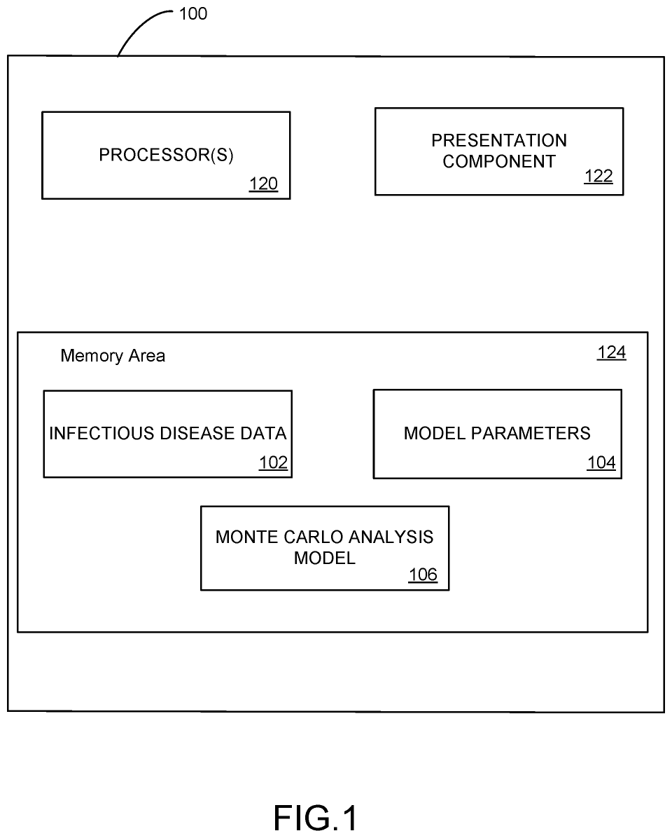

With reference now to FIG. 1, a block diagram of a computing device 100 for creating unique disease timelines for modeling an efficacy of a screening and testing strategy for an infectious disease is provided. The computing device 100 includes one or more processors 120, one or more presentation components 122 and a memory area 124. The disclosed examples associated with the computing device 100 can be practiced by a variety of computing devices, including personal computers, laptops, smart phones, mobile tablets, hand-held devices, consumer electronics, specialty computing devices, etc. The disclosed examples are also practiced in distributed computing environments, where tasks are performed by remote-processing devices that are linked through a communications network, for example, via distributed computing environment hosts cloud synthetics services. Further, while the computing device 100 is depicted as a seemingly single device, in one embodiment, multiple computing devices work together and share the depicted device resources. For instance, in one embodiment, the memory area 124 is distributed across multiple devices, the processor(s) 120 provided are housed on different devices, and so on.

In one embodiment, the processor(s) 120 includes any quantity of processing units that read data from various entities, such as the memory area 124. Specifically, the processor(s) 120 are programmed to execute computer-executable instructions for implementing aspects of the disclosure. In one embodiment, the instructions are performed by the processor, by multiple processors within the computing device 100, or by a processor external to the computing device 100. In some examples, the processor(s) 120 are programmed to execute instructions such as those illustrated in the flowcharts discussed below and depicted in the accompanying drawings. Moreover, in some examples, the processor(s) 120 represent an implementation of analog techniques to perform the operations described herein. For example, the operations are performed by an analog client computing device 100 and/or a digital client computing device 100.

The presentation component(s) 122 present data indications to a user of the computing device 100 or to another device. Exemplary presentation components include a display device, speaker, printing component, vibrating component, etc. One skilled in the art will understand and appreciate that computer data is presented in a number of ways, such as visually in a graphical user interface (GUI), audibly through speakers, wirelessly between the computing device 100, across a wired connection, or in other ways.

The memory area 124 stores infectious disease data 102, model parameters 104, and Monte Carlo analysis model 106. The infectious disease data 102 includes information corresponding to infectious diseases including real-world data from studies of the infectious diseases and events for a disease timeline for an infectious disease. The events for an infectious disease comprise one or more of the following: disease exposure, symptom onset, severe symptom onset, and an end of contagious period. The model parameters 104 includes a type of test being performed (e.g., RT-PCR) and corresponding test parameters, for example a length of time for the contagious period, a first point in time on a model wherein sensitivity is at a maximum, and a second point in time after the first point in time on the model that the sensitivity is at a minimum. The Monte Carlo analysis model 106 is a software statistical analysis tool that provides efficacy metrics of a screening an testing strategy for an infectious disease and produces a measure of performance for a diagnostic test that is specific to a simulated infected person's unique disease timeline.

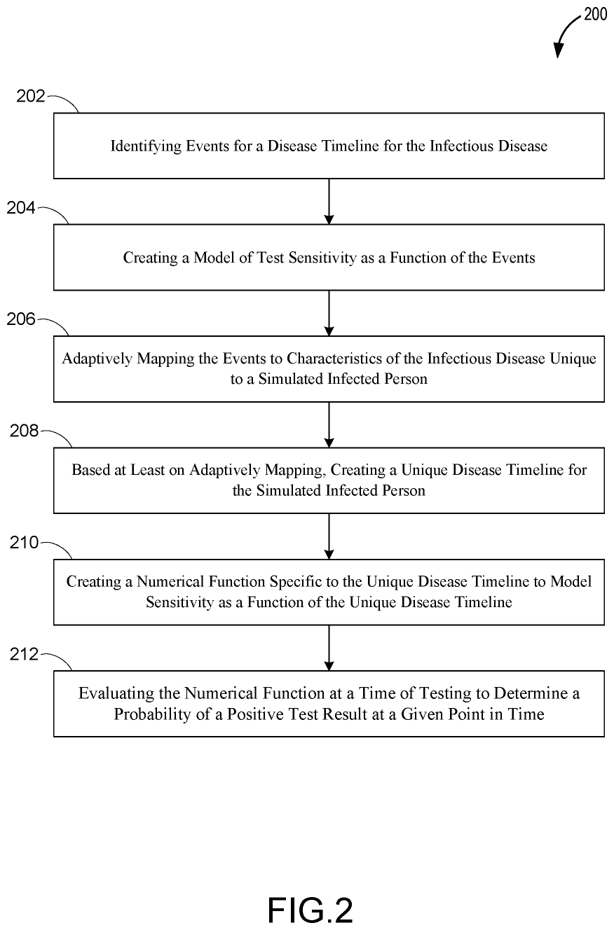

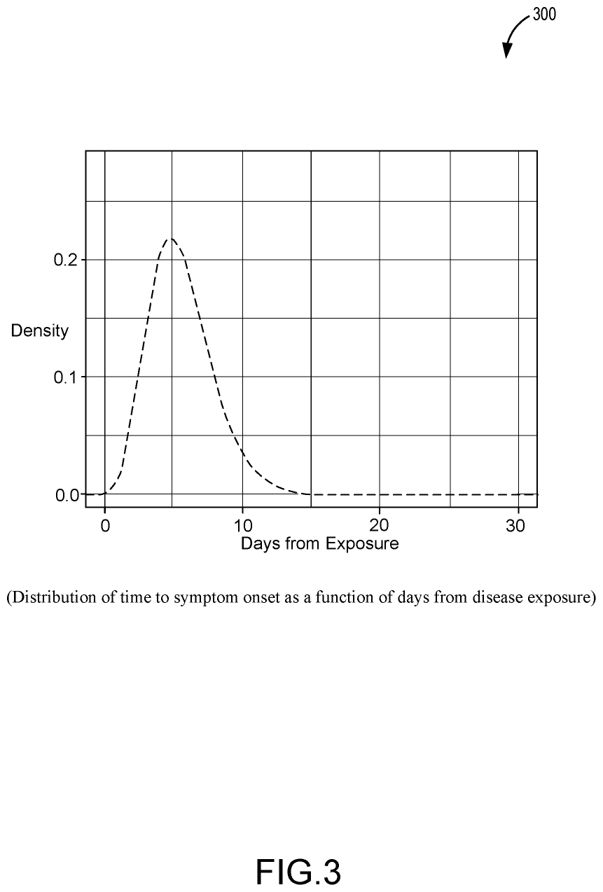

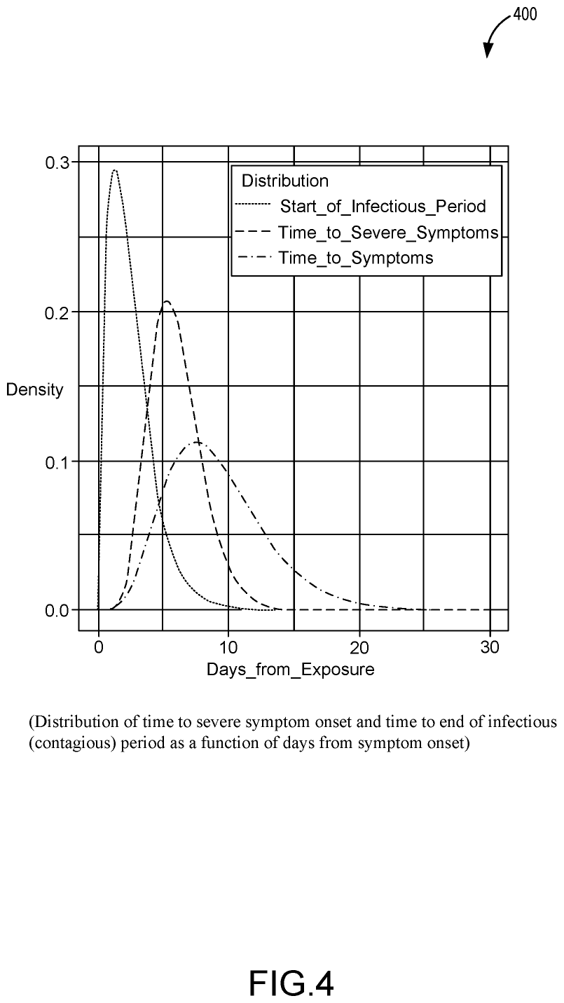

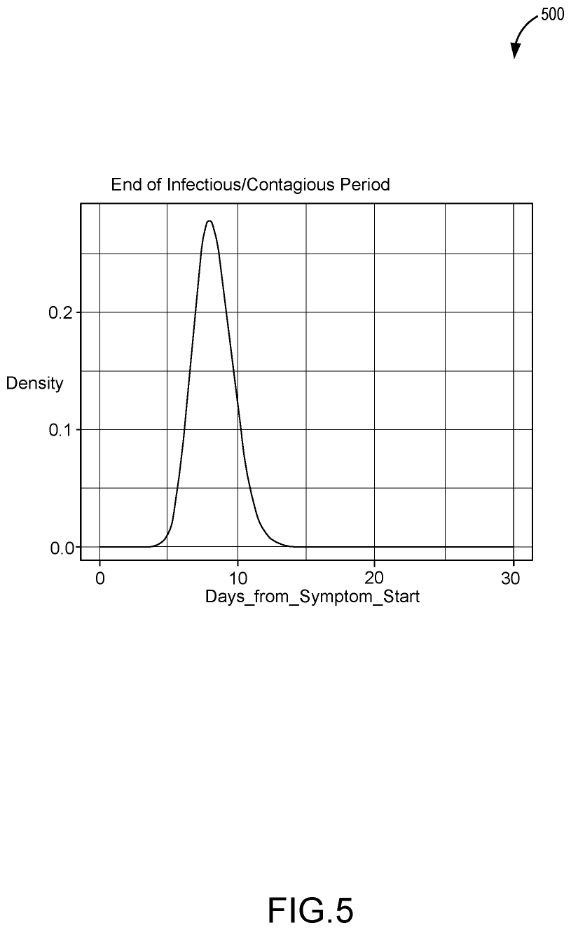

With reference now to FIG. 2, a block diagram illustrating a process 200 for creating unique disease timelines for the Monte Carlo analysis model 106 to obtain efficacy metrics of a screening and testing strategy for an infectious disease. To set up a simulation for the Monte Carlo analysis model 106, a timeline of an infectious disease is first be established and at 202, events for a disease timeline for an infectious disease are identified. For example, using the information for the infectious disease from the infectious disease data 102, the disease timeline is separated into several events (e.g., time points), such as disease exposure, symptom onset, severe symptom onset, and an end of contagious period. At 204, a model of test sensitivity as a function of the events is created. In one example, the model of test sensitivity is created as a function of the events, such that “y” represents test performance in terms of sensitivity and “x” represents the events, which do not yet have numeric values. In one example, the model parameters 104 for the infectious disease is accessed to identify parameters to be set for the infectious disease timeline/model. In this example, the model parameters 104 indicate that the disease exposure is set to occur at time 0 and all other events are modeled as Gamma distributions. Further, the model parameters 104 indicate that a maximum sensitivity of RT-PCR testing is set to be 99%, and a specificity (true negative rate or proportion of uninfected population that test negative) is set to be 99%. As shown in FIG. 3, a distribution for time to symptom onset for the simulated infected person is provided in diagram 300. As shown in FIG. 4, a time to onset of severe symptoms and a time to end of the contagious period are modeled as a function of time from symptom onset is provided in diagram 400. As shown in FIG. 5 a probability density of time from symptom onset to end of contagious period is provided in diagram 500.

With reference back to FIG. 2, at 206, the events are adaptively mapped to characteristics of the infectious disease that are unique to a simulated infected person. For example, as explained above, the present disclosure creates unique disease timelines for each simulated infected person (e.g., each passenger) unlike using a generic function that is applied to all individuals as conventional screening and testing strategies. Thus, time points for the simulated infected person are mapped to the events of the infectious disease to create numeric values for “x” in the test sensitivity model. That is, a value for “x” for each event represents days from initial infection for the particular event. In one example, the time points/events unique to the simulated infected person are obtained by random selection of time points from the infectious disease data 102. That is, data such as time points/events for the simulated infected person are based on real-world data and studies of infected persons of the infectious disease. Thus, in some examples, data for the simulated infected person is based on data from one or more real-world infected persons captured in the infectious disease data 102. In some examples, the data for the simulated infected person uses data from a particular location and/or data from a defined period of time (historic data or most current data).

At 208, a unique disease timeline for the simulated infected person is created based at least on adaptively mapping the events to characteristics of the infectious disease unique to a simulated infected person. The unique disease timeline includes numerical values for each of the events with the numerical values representing days from an initial infection for the simulated infected person. Further an individual test performance trajectory for the simulated infected person based at least on the events in the unique disease timeline is created using cubic splines in order to describe test sensitivity as a function of days from initial infection. At 210, a numerical function specific to the unique disease timeline to model sensitivity as a function of the unique disease timeline is created. At 212, the numerical function at a time of testing is evaluated to determine a probability of a positive test result at a given point in time.

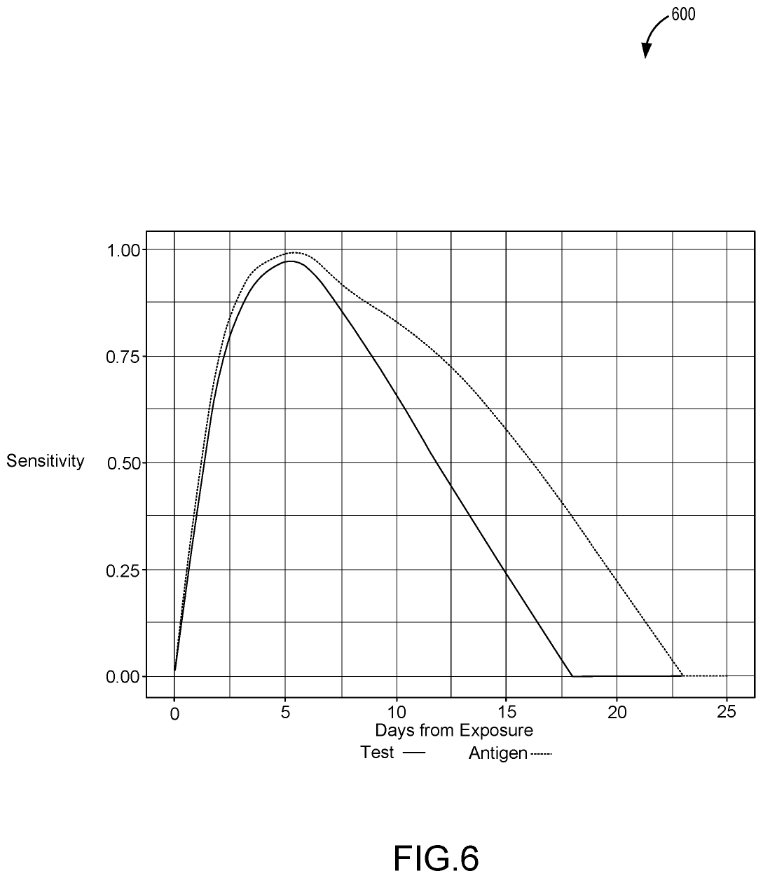

With reference now to FIG. 6, the end of the contagious period is modeled as a function of time from symptom onset in diagram 600. The curves shown in FIG. 6 are an example for a “typical” disease timeline with symptom onset on day 5 post-exposure and contagious period ending on day 13. FIG. 6 shows the unique effectiveness of RT-PCR and antigen testing, measured in terms of sensitivity (true positive rate or proportion of true cases that test positive), which was modeled as a function of time from exposure. This unique test performance function is fitted to each simulated infected person's disease timeline using cubic splines such that the test sensitivity peaked at the day of symptom onset and trailed off to zero at 10 days past the end of the contagious period for RT-PCR or 3 days past the end of the contagious period for antigen tests.

Efficacy metrics described in the results below are obtained through a Monte Carlo simulation-based analysis using the Monte Carlo analysis model 106. However, when applying the Monte Carlo simulation-based analysis using each of the respective unique timelines for the simulated infected persons, the examples described herein make assumptions based in part on the infectious disease data 102 and the model parameters 104. For example: not all infected persons will develop severe symptoms, 30% of cases are asymptomatic, asymptomatic travelers are infectious, 15% of infected persons will develop moderate/severe symptoms, would-be travelers will not travel if symptoms are moderate/severe, all travelers without moderate/severe symptoms will attempt to travel, passengers have disease prevalence rate equivalent to departure country, the average contagious period is 12 days, for all infected passengers, a departure time is randomly placed between a time of exposure and an end of the contagious period with a uniform distribution, passengers that are infected but are no longer contagious at departure time are not considered in this analysis, no new cases will develop after time of departure, all tests are performed independent regardless of whether they are the same or different test types, all passengers will self-quarantine until final screening test is administered such that no new infections are acquired after arrival and that all passengers will be fully compliant with any length of quarantine imposed unless otherwise noted, and travel time is four hours.

In addition, the Monte Carlo analysis model 106 assumes that would-be travelers will not travel if they are experiencing severe symptoms and will consequently self-select out of the travel system. All other infected would-be travelers who may be experiencing no symptoms or mild symptoms will attempt to travel. The Monte Carlo analysis model 106 further assumes that no new cases will develop after the time of departure, so infections acquired during the travel journey are not included. These assumptions are varied to model different regions' behaviors or virus variants, but for the examples described herein, these particular variables are locked.

Results of the Monte Carlo simulation-based analysis are reported in terms of sensitivity, specificity, positive predictive value (PPV), and negative predicted value (NPV). Definitions and interpretations are as follows:

Sensitivity—the proportion of true positive cases who test positive using the screening test of interest, also known as the true positive rate.

Specificity—the proportion of true negatives (uninfected passengers) who test negative using the screening test of interest, also known as the true negative rate.

Positive Predictive Value (PPV)—the proportion of passengers who test positive using the screening test of interest who are truly infected with the disease.

Negative Predictive Value (NPV)—the proportion of passengers who test negative using the screening test of interest who are truly uninfected.

Sensitivity is estimated using the percentage of the simulated infected passengers who tested positive using the screening test of interest. Specificity is estimated using the percentage of simulated uninfected passengers who tested negative using the screening test of interest. PPV is estimated using the percentage of all simulated passengers who tested positive using the screening test of interest who truly were infected. NPV is estimated using the percentage of all simulated passengers who tested negative using the screening test of interest who were truly uninfected. Cases who were beyond the end of the contagious period at the time of testing were excluded from the computations. Note that PPV and NPV are dependent on the disease prevalence of the country of interest, so they will vary between countries. However, sensitivity and specificity are not, so they should be roughly equal for equivalent testing methods with different countries of origin, subject to some variability due to the stochastic nature of the Monte Carlo analysis.

The simulated persons on each flight are assumed to have disease prevalence rates equivalent to that of the departure country. Prevalence for each country was calculated using the following formula:

Prevalence=(daily new case incidence rate per 100,000 population)*(1+undetected case multiplier)*(average contagious period)/100,000

This metric provides the ability to compare the prevalence of the origin country, and more importantly the destination country, to the prevalence of passengers who have been subjected to a specific screening or quarantine strategy. Results Table 1 (below) displays the prevalence values for each risk level included in this analysis.

TABLE 1

| Risk Level | Prevalence |
|---|---|
| Very Low | 0.00003 |
| Low | 0.00033 |
| Medium | 0.0033 |
| High | 0.0105 |
| Very High | 0.03 |

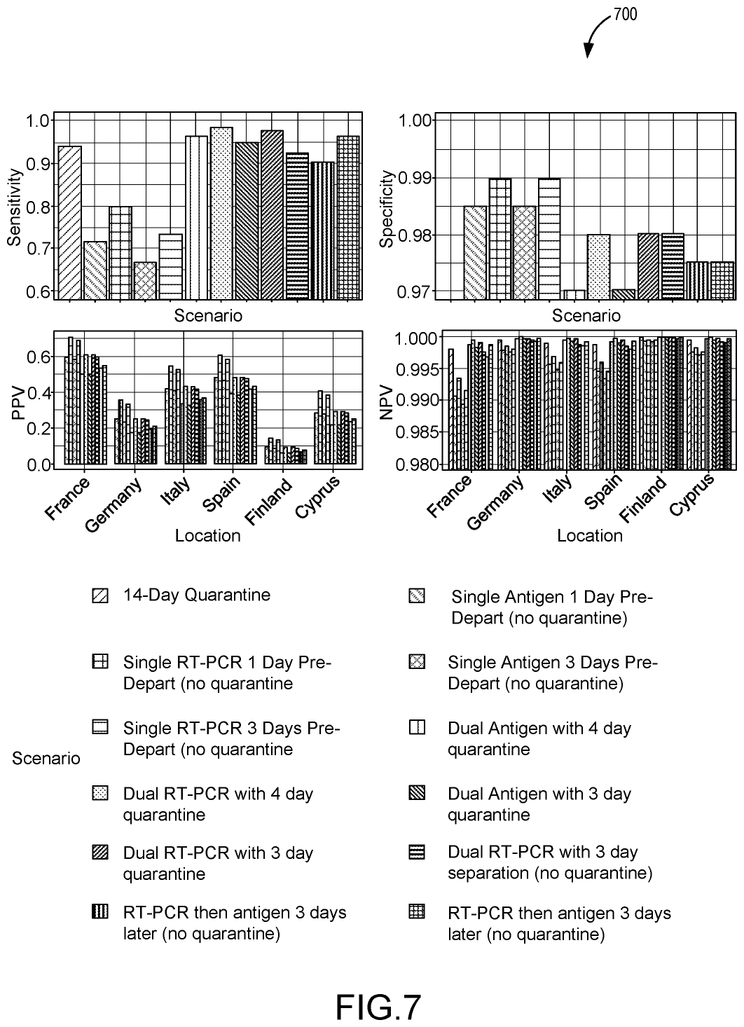

FIG. 7 provides a summary 700 of results for the 4 major metrics of interest: sensitivity (A), specificity (B), PPV (C), and NPV (D). Sensitivity and specificity do not depend on prevalence and therefore do not change from country to country. Note that for the dual testing strategies, the sensitivity is higher than single test methods, but the specificity is lower. Conducting two tests allows for two chances for a false positive, so the false positive rate is higher. Further, PPV is low for countries with low prevalence and higher for countries with higher prevalence. This is due to the fact that there are so few true cases in countries with low prevalence that most positive test results are false positives.

Based on the herein described model, about 94% of cases will no longer be infectious after a 14-day quarantine (estimated sensitivity for quarantine=94%). When testing pre-departure without quarantine, the closer the test is conducted to the departure time, the better the sensitivity (the more cases that can be detected). Dual testing scenarios perform much better than single tests in terms of sensitivity. Dual testing strategies with a short quarantine (3 to 4 days) perform the best of all the strategies considered, such that the sensitivity is similar to or perhaps even better than a 14-day quarantine based on these model results. This quarantine can be conducted either pre-departure or post-arrival as long as a dual testing strategy is employed such that each traveler is tested prior to the start of the quarantine period and after the completion of the short quarantine period. Even without any quarantine, dual testing strategies can perform almost as well as a 14-day quarantine in terms of sensitivity/NPV.

Further, using post-screening prevalence for a travel journey between a high prevalence country and a lower prevalence country, travelers begin with the prevalence of the origin country and the prevalence of the population of travelers is reduced by screening out cases using the various screening strategies. Any screening strategy which provides a reduction of prevalence to a level below the destination prevalence provides confidence that the incoming travelers are no more likely to be infected than the existing residents of the destination country.

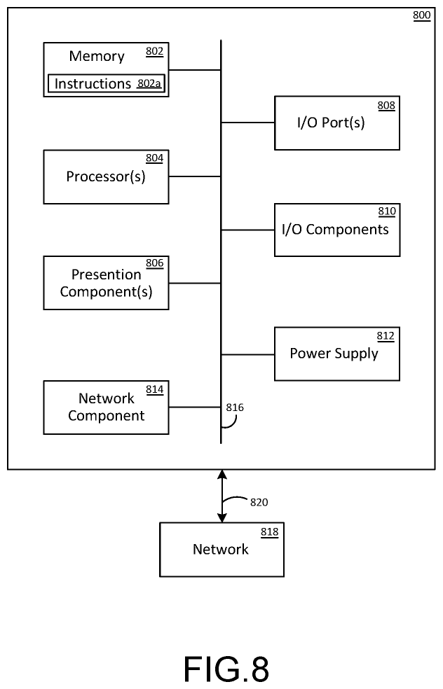

With reference now to FIG. 8, a block diagram of the computing device 800 suitable for implementing various aspects of the disclosure is described. In some examples, the computing device 800 includes one or more processors 804, one or more presentation components 806 and the memory 802. The disclosed examples associated with the computing device 800 are practiced by a variety of computing devices, including personal computers, laptops, smart phones, mobile tablets, hand-held devices, consumer electronics, specialty computing devices, etc. Distinction is not made between such categories as “workstation,” “server,” “laptop,” “hand-held device,” etc., as all are contemplated within the scope of FIG. 8 and the references herein to a “computing device.” The disclosed examples are also practiced in distributed computing environments, where tasks are performed by remote-processing devices that are linked through a communications network. Further, while the computing device 800 is depicted as a seemingly single device, in one example, multiple computing devices work together and share the depicted device resources. For instance, in one example, the memory 802 is distributed across multiple devices, the processor(s) 804 provided are housed on different devices, and so on.

In one example, the memory 802 includes any of the computer-readable media discussed herein. In one example, the memory 802 is used to store and access instructions 802a configured to carry out the various operations disclosed herein. In some examples, the memory 802 includes computer storage media in the form of volatile and/or nonvolatile memory, removable or non-removable memory, data disks in virtual environments, or a combination thereof. In one example, the processor(s) 804 includes any quantity of processing units that read data from various entities, such as the memory 802 or input/output (I/O) components 810. Specifically, the processor(s) 804 are programmed to execute computer-executable instructions for implementing aspects of the disclosure. In one example, the instructions are performed by the processor, by multiple processors within the computing device 800, or by a processor external to the computing device 800. In some examples, the processor(s) 804 are programmed to execute instructions such as those illustrated in the flow charts discussed below and depicted in the accompanying drawings.

The presentation component(s) 806 present data indications to an operator or to another device. In one example, presentation components 806 include a display device, speaker, printing component, vibrating component, etc. One skilled in the art will understand and appreciate that computer data is presented in a number of ways, such as visually in a graphical user interface (GUI), audibly through speakers, wirelessly between the computing device 800, across a wired connection, or in other ways. In one example, presentation component(s) 806 are not used when processes and operations are sufficiently automated that a need for human interaction is lessened or not needed. I/O ports 808 allow the computing device 800 to be logically coupled to other devices including the I/O components 810, some of which is built in. Implementations of the I/O components 910 include, for example but without limitation, a microphone, keyboard, mouse, joystick, game pad, satellite dish, scanner, printer, wireless device, etc.

The computing device 800 includes a bus 816 that directly or indirectly couples the following devices: the memory 802, the one or more processors 804, the one or more presentation components 806, the input/output (I/O) ports 808, the I/O components 810, a power supply 812, and a network component 814. The computing device 800 should not be interpreted as having any dependency or requirement related to any single component or combination of components illustrated therein. The bus 816 represents one or more busses (such as an address bus, data bus, or a combination thereof). Although the various blocks of FIG. 8 are shown with lines for the sake of clarity, some implementations blur functionality over various different components described herein.

In some examples, the computing device 800 is communicatively coupled to a network 818 using the network component 814. In some examples, the network component 814 includes a network interface card and/or computer-executable instructions (e.g., a driver) for operating the network interface card. In one example, communication between the computing device 800 and other devices occur using any protocol or mechanism over a wired or wireless connection 820. In some examples, the network component 814 is operable to communicate data over public, private, or hybrid (public and private) using a transfer protocol, between devices wirelessly using short range communication technologies (e.g., near-field communication (NFC), Bluetooth® branded communications, or the like), or a combination thereof.

Although described in connection with the computing device 800, examples of the disclosure are capable of implementation with numerous other general-purpose or special-purpose computing system environments, configurations, or devices. Implementations of well-known computing systems, environments, and/or configurations that are suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, VR devices, holographic device, and the like. Such systems or devices accept input from the user in any way, including from input devices such as a keyboard or pointing device, via gesture input, proximity input (such as by hovering), and/or via voice input.

Implementations of the disclosure are described in the general context of computer-executable instructions, such as program modules, executed by one or more computers or other devices in software, firmware, hardware, or a combination thereof. In one example, the computer-executable instructions are organized into one or more computer-executable components or modules. Generally, program modules include, but are not limited to, routines, programs, objects, components, and data structures that perform particular tasks or implement particular abstract data types. In one example, aspects of the disclosure are implemented with any number and organization of such components or modules. For example, aspects of the disclosure are not limited to the specific computer-executable instructions or the specific components or modules illustrated in the figures and described herein. Other examples of the disclosure include different computer-executable instructions or components having more or less functionality than illustrated and described herein. In implementations involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. In one example, computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium used to store information for access by a computing device. In contrast, communication media typically embody computer readable instructions, data structures, program modules, or the like in a modulated data signal such as a carrier wave or other transport mechanism and include any information delivery media.

The examples disclosed herein are described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples are practiced in a variety of system configurations, including personal computers, laptops, smart phones, mobile tablets, hand-held devices, consumer electronics, specialty computing devices, etc. The disclosed examples are also practiced in distributed computing environments, where tasks are performed by remote-processing devices that are linked through a communications network.

An example method for creating unique disease timelines for modeling an efficacy of a screening and testing strategy for an infectious disease comprises: identifying events for a disease timeline for the infectious disease, the events comprising disease exposure, a symptom onset, a severe symptom onset, and an end of contagious period; creating a model of test sensitivity as a function of the events; adaptively mapping the events to characteristics of the infectious disease unique to a simulated infected person; based at least on adaptively mapping, creating a unique disease timeline for the simulated infected person; and creating a numerical function specific to the unique disease timeline to model sensitivity as a function of the unique disease timeline.

An example system for creating unique disease timelines for modeling an efficacy of a screening and testing strategy for an infectious disease comprises: identifying events for a disease timeline for the infectious disease, the events comprising disease exposure, a symptom onset, a severe symptom onset, and an end of contagious period; creating a model of test sensitivity as a function of the events; adaptively mapping the events to characteristics of the infectious disease unique to a simulated infected person; based at least on adaptively mapping, creating a unique disease timeline for the simulated infected person; and creating a numerical function specific to the unique disease timeline to model sensitivity as a function of the unique disease timeline.

An example computer-readable media comprises computer-executable instructions that, when executed by one or more processors, cause the one or more processors to perform the following operations: identifying events for a disease timeline for an infectious disease, the events comprising disease exposure, a symptom onset, a severe symptom onset, and an end of contagious period; creating a model of test sensitivity as a function of the events; adaptively mapping the events to characteristics of the infectious disease unique to a simulated infected person; based at least on adaptively mapping, creating a unique disease timeline for the simulated infected person; and creating a numerical function specific to the unique disease timeline to model sensitivity as a function of the unique disease timeline.

Alternatively, or in addition to the other examples described herein, examples include any combination of the following:
the unique disease timeline provides numerical values for each of the events, the numerical values representing days from an initial infection for the simulated infected person;
creating, using cubic splines, an individual test performance trajectory based at least on the events in the unique disease timeline;
based at least on the numerical function, determining a probability of a positive test result for the simulated infected person at a particular point in time;
creating the model of test sensitivity as the function of the events comprises providing parameters for the model;
the parameters comprise: a type of test and corresponding test parameters, a length of time for the contagious period, a first point in time on the model that sensitivity is at a maximum, and a second point in time after the first point in time on the model that the sensitivity is at a minimum;
evaluating the numerical function at a time of testing is to determine a probability of a positive test result at a given point in time;
using the Monte Carlo Analysis model to determine an efficacy of the screening test of interest based on a percentage of simulated infected passengers who tested positive using the screening test of interest; and
accessing a database comprising real world data of the infectious disease, and wherein the characteristics of the infectious disease unique to the simulated infected person are from one or more infected persons from the real world data.

When introducing elements of aspects of the disclosure or the implementations thereof, the articles “a,” “an,” “the,” and “said” are intended to mean that there are one or more of the elements. The terms “comprising,” “including,” and “having” are intended to be inclusive and mean that there could be additional elements other than the listed elements. The term “implementation” is intended to mean “an example of” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

Although particular aspects and embodiments have been shown and described, it should be understood that the above discussion is not intended to limit the scope of these embodiments. While embodiments and variations of the many aspects of the invention have been disclosed and described herein, such disclosure is provided for purposes of explanation and illustration only. Thus, various changes and modifications may be made without departing from the scope of the claims.

Where methods described above indicate certain events occurring in certain order, those of ordinary skill in the art having the benefit of this disclosure would recognize that the ordering may be modified and that such modifications are in accordance with the variations of the present disclosure. Additionally, parts of methods may be performed concurrently in a parallel process when possible, as well as performed sequentially. In addition, more steps or less steps of the methods may be performed.

Accordingly, embodiments are intended to exemplify alternatives, modifications, and equivalents that may fall within the scope of the claims.

Although certain illustrative embodiments and methods have been disclosed herein, it can be apparent from the foregoing disclosure to those skilled in the art that variations and modifications of such embodiments and methods can be made without departing from the true spirit and scope of this disclosure. Many other examples exist, each differing from others in matters of detail only. Accordingly, it is intended that this disclosure be limited only to the extent required by the appended claims and the rules and principles of applicable law.

What is claimed is:

1. A method for creating unique disease timelines for modeling an efficacy of a screening and testing strategy for an infectious disease, the method comprising:
identifying events for a disease timeline for the infectious disease, the events comprising disease exposure, a symptom onset, a severe symptom onset, and an end of contagious period;
creating a model of test sensitivity as a function of the events;
adaptively mapping the events to characteristics of the infectious disease unique to a simulated infected person;
based at least on adaptively mapping, creating a unique disease timeline for the simulated infected person; and
creating a numerical function specific to the unique disease timeline to model sensitivity as a function of the unique disease timeline.

2. The method of claim 1, wherein the unique disease timeline provides numerical values for each of the events, the numerical values representing days from an initial infection for the simulated infected person.

3. The method of claim 2, further comprising creating, using cubic splines, an individual test performance trajectory based at least on the events in the unique disease timeline.

4. The method of claim 1, further comprising, based at least on the numerical function, determining a probability of a positive test result for the simulated infected person at a particular point in time.

5. The method of claim 1, wherein creating the model of test sensitivity as the function of the events comprises providing parameters for the model.

6. The method of claim 5, wherein the parameters comprise: a type of test and corresponding test parameters, a length of time for the contagious period, a first point in time on the model that sensitivity is at a maximum, and a second point in time after the first point in time on the model that the sensitivity is at a minimum.

7. The method of claim 1, further comprising:
evaluating the numerical function at a time of testing is to determine a probability of a positive test result at a given point in time; and
using the Monte Carlo Analysis model to determine an efficacy of the screening test of interest based on a percentage of simulated infected passengers who tested positive using the screening test of interest.

8. The method of claim 1, further comprising accessing a database comprising real world data of the infectious disease, and wherein the characteristics of the infectious disease unique to the simulated infected person are from one or more infected persons from the real world data.

9. A system for creating unique disease timelines for modeling an efficacy of a screening and testing strategy for an infectious disease, the system comprising:
a database;
one or more processors programmed to perform the following operations:
identifying events for a disease timeline for the infectious disease, the events comprising disease exposure, a symptom onset, a severe symptom onset, and an end of contagious period;
creating a model of test sensitivity as a function of the events;
adaptively mapping the events to characteristics of the infectious disease unique to a simulated infected person;
based at least on adaptively mapping, creating a unique disease timeline for the simulated infected person; and
creating a numerical function specific to the unique disease timeline to model sensitivity as a function of the unique disease timeline.

10. The system of claim 9, wherein the unique disease timeline provides numerical values for each of the events, the numerical values representing days from an initial infection for the simulated infected person.

11. The system of claim 10, wherein the one or more processors are further programmed to perform the following operation, creating, using cubic splines, an individual test performance trajectory based at least on the events in the unique disease timeline.

12. The system of claim 9, wherein the one or more processors are further programmed to perform the following operation based at least on the numerical function, determining a probability of a positive test result for the simulated infected person at a particular point in time.

13. The system of claim 9, wherein creating the model of test sensitivity as the function of the events comprises providing parameters comprising a type of test and corresponding test parameters, a length of time for the contagious period, a first point in time on the model that sensitivity is at a maximum, and a second point in time after the first point in time on the model that the sensitivity is at a minimum.

14. The system of claim 13, wherein the database comprises real world data of the infectious disease, and wherein the one or more processors are further programmed to perform the following operation, accessing, from the database, the real world data of the infectious disease, and wherein the characteristics of the infectious disease unique to the simulated infected person are from one or more infected persons from the real world data.

15. A computer-readable media comprising computer-executable instructions that, when executed by one or more processors, cause the one or more processors to perform the following operations:
identifying events for a disease timeline for an infectious disease, the events comprising disease exposure, a symptom onset, a severe symptom onset, and an end of contagious period;
creating a model of test sensitivity as a function of the events;
adaptively mapping the events to characteristics of the infectious disease unique to a simulated infected person;
based at least on adaptively mapping, creating a unique disease timeline for the simulated infected person; and
creating a numerical function specific to the unique disease timeline to model sensitivity as a function of the unique disease timeline.

16. The computer-readable media of claim 15, wherein the unique disease timeline provides numerical values for each of the events, the numerical values representing days from an initial infection for the simulated infected person.

17. The computer-readable media of claim 16, wherein the computer-executable instructions further cause the one or more processors to perform the following operation, creating, using cubic splines, an individual test performance trajectory based at least on the events in the unique disease timeline.

18. The computer-readable media of claim 15, wherein the computer-executable instructions further cause the one or more processors to perform the following operation, based at least on the numerical function, determining a probability of a positive test result for the simulated infected person at a particular point in time.

19. The computer-readable media of claim 15, wherein creating the model of test sensitivity as the function of the events comprises providing parameters comprising a type of test and corresponding test parameters, a length of time for the contagious period, a first point in time on the model that sensitivity is at a maximum, and a second point in time after the first point in time on the model that the sensitivity is at a minimum.

20. The computer-readable media of claim 19, wherein the computer-executable instructions further cause the one or more processors to perform the following operation, accessing, from a database, real world data of the infectious disease, and wherein the characteristics of the infectious disease unique to the simulated infected person are from one or more infected persons from the real world data.
